(19) United States
(12) Patent Application Publication
(10) Pub. No.: US 2003/0211138 A1
(43) Pub. Date: Nov. 13, 2003

(54) Temperature control system for cutaneous photodynamic therapy

(75) Inventors: Jeffrey L. Conroy, Rumford, RI (US); Nabil M. Lawandy, North Kingstown, RI (US); Timothy J. Driscoll, Pawtucket, RI (US); Charles M. Zepp, Hardwick, MA (US)

Correspondence Address: HARRINGTON & SMITH, LLP, 4 RESEARCH DRIVE, SHELTON, CT, 06484-6212, US

(73) Assignee: Spectra Systems Corporation

(21) Appl. No.: 10/370,907

(22) Filed: Feb. 20, 2003

Related U.S. Application Data
(60) Provisional application No. 60/359,254, filed on Feb. 20, 2002.

(51) Int. Cl. A61K 31/765; A61N 1/30; A61K 9/70

(52) U.S. Cl. 424/449; 424/78.32; 604/20

(57) ABSTRACT

A method is disclosed for limiting temperature increase of a treatment area during photodynamic therapy (PDT), whereby temperature sensitive materials in the form of a skin patch or compound are applied over the treatment area. The temperature sensitive materials are optically changing from substantially transmissive to at least one of substantially scattering, absorbing or reflecting at wavelengths used for the PDT, the change occurring upon surpassing a predetermined temperature. In preferred embodiments, the optical changes are reversible as the temperature of the temperature sensitive compounds decrease below a predetermined temperature.

### CROSS REFERENCE TO RELATED APPLICATION

This application is filed under 35 U.S.C. §111(a) and claims priority under 35 U.S.C. §119(e) to U.S. Provisional Patent Application No. 60/359,254, filed Feb. 20, 2002, and entitled “Temperature Control System for Cutaneous Photodynamic Therapy.”

### TECHNICAL FIELD

These teachings relate generally to the prevention of overheating of a target tissue during photodynamic therapy through the application of temperature sensitive materials.

### BACKGROUND

Recent advances in medicine have introduced photodynamic therapy (PDT) as a method for treatment of a variety of illnesses. PDT is based upon the discovery that the introduction of certain chemicals known generally as photosensitizing agents into one-celled organisms can kill the organisms once they have been exposed to a particular type of light. Relying upon this discovery, effective treatments for certain types of cancer have been developed. Skin cancer is among the types of cancer where PDT has been determined to provide an effective method of treatment.

In a typical application of PDT, a photosensitizing agent is injected into the bloodstream. This agent is absorbed by cells throughout the body, however, the agent remains in the cancerous cells for a longer period of time than it does in the healthy cells of the body. This phenomenon is relied upon to prepare the cancerous cells for treatment. Once levels of the photosensitizing agent have subsided within the surrounding healthy tissue, laser light is used to expose the photosensitized cancerous cells. This laser light produces chemical changes within the photosensitized cancerous cells that ultimately kill the diseased tissue. This method of treating skin cancer has a number of advantages, including minimal collateral damage to healthy tissue. Of course, careful timing of the exposure to laser light, and accurate control of the energy deposited by the laser are required to ensure that minimal collateral damage occurs.

U.S. Pat. No. 6,428,811 B1, issued Aug. 6, 2002 to West et al., entitled “Temperature Sensitive Polymer/Nanoshell Composites for Photothermally Modulated Drug Delivery” discloses the use of absorbed energy to trigger the delivery of a chemical isolated within a thermally sensitive polymer-particle composite. The nanoshells disclosed therein are combined with a temperature sensitive material to provide an implantable or injectable material for modulated drug delivery. This patent does not disclose or suggest methods for controlling energy deposition in a target tissue.

Although electronic controls can provide for accurate control of the energy deposited by the laser, the effect on the target tissue is usually only subject to prediction based upon clinical knowledge. The actual effect on each target tissue may vary depending upon a variety of factors including water content, pore structures and other factors that dissipate thermal energy from the skin. Failure to adequately control the energy deposition, and/or the treatment of abnormally sensitive skin, can result in overheating the target tissue, and result in detrimental side effects including pain and potential damage to healthy tissue.

### SUMMARY OF THE PREFERRED EMBODIMENTS

The foregoing and other problems are overcome, and other advantages are realized, in accordance with the presently preferred embodiments of these teachings.

Disclosed herein are methods and apparatus for controlling the energy deposition or temperature of tissue in a treatment area during administration of a therapeutic light, such as in photodynamic therapy.

The invention disclosed herein makes use of temperature sensing materials that absorb laser energy once a target temperature has been reached. In a preferred embodiment, a polymer, or mixture of polymers, is applied to the target area as a skin patch. The polymer, or mixture of polymers are transparent, or substantially transparent at ambient temperatures. Laser light, or a similar energy form suited for photodynamic therapy, is directed through the skin patch to the target tissue in a manner called for by the treatment regimen. As tissue in the target area absorbs energy from the therapeutic light, its temperature begins to rise. Likewise, the temperature of the skin patch begins to rise. Once heated above a threshold temperature, the polymer or polymer mixture exhibits an optical change, and the polymer or polymer mixture becomes reflective, absorptive or scattering of the therapeutic light, thus reducing further energy deposition. As the energy absorbed by the tissue dissipates, the temperature of the target area decreases, and the skin patch again becomes transmissive or substantially transmissive, thereby permitting treatment to continue relatively unabated. Through the use of an appropriate skin patch, the actual temperature of the target tissue can be carefully controlled, harmful side effects from excess exposure to the energy source can be prevented, and treatment can be completed without interruption.

In another embodiment, the temperature sensing materials are dispersed over an intermediate transfer mechanism, such as a ventilated bandage. This ventilated bandage provides for ease of application and removal, and permits heat dissipation processes of the skin to occur unimpeded.

In a further embodiment, the temperature sensing materials are distributed in a cream or paint for application directly to the skin.

### BRIEF DESCRIPTION OF THE DRAWINGS

The foregoing and other aspects of these teachings are made more evident in the following Detailed Description of the Preferred Embodiments, when read in conjunction with the attached Drawing Figures, wherein:

FIG. 1 is a cross sectional diagram of a skin patch, applied over a portion of skin which is to be treated with photodynamic therapy;

FIG. 2 is a cross sectional diagram of a skin patch, applied over a portion of skin, where the patch has been activated by laser energy used in the photodynamic therapy;

FIG. 3 illustrates a first preferred embodiment of the skin patch;

FIG. 4 illustrates a second preferred embodiment of the skin patch;

FIG. 5 illustrates a customized skin patch;

FIG. 6 illustrates a two layer skin patch;

FIG. 7 is a graph that depicts typical light transmission for a formulation over a range of wavelengths;

FIG. 8 is a graph that depicts an optical transmission of a mixture for a specific wavelength as a function of temperature;

FIG. 9 is a graph that depicts the effect of varying concentration of NaCl in a mixture on optical transmission transition temperature;

FIG. 10 is a graph that depicts the effect of varying concentration of DMF in a mixture on optical transmission transition temperature;

FIG. 11 is a graph that depicts the effect of varying concentration of DMSO in a mixture on optical transmission transition temperature; and,

FIG. 12 shows the chemical structures of thermochromic compositions of polymer mixtures used in combination with color formers and Lewis Acids.

### DETAILED DESCRIPTION OF THE PREFERRED EMBODIMENTS

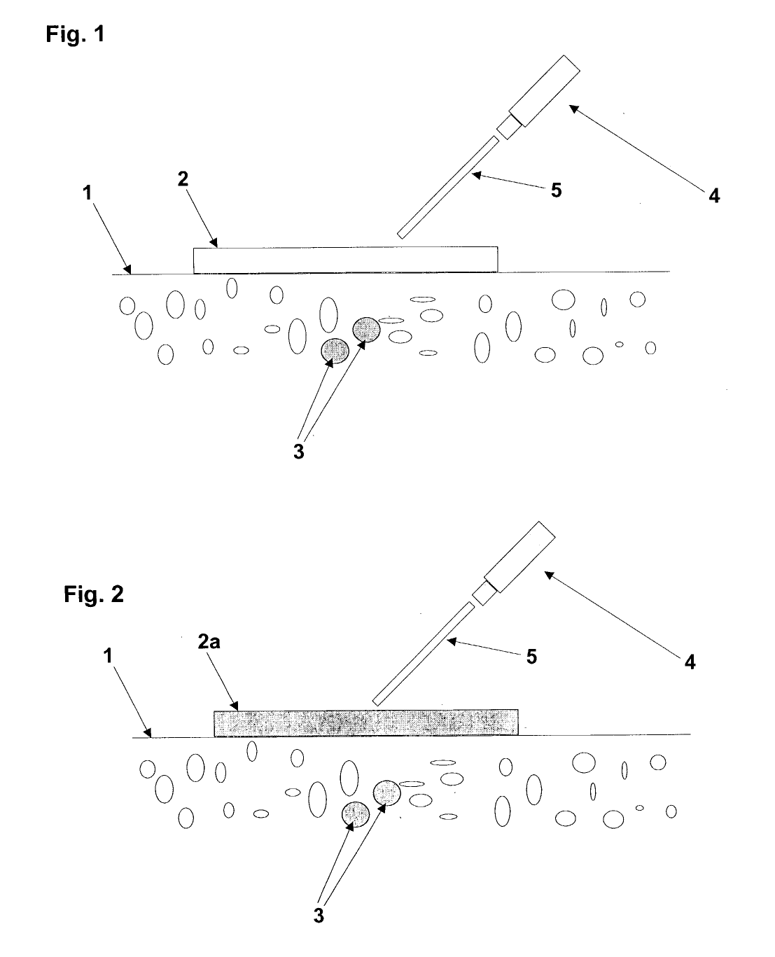

FIG. 1 illustrates the application of an epidermal patch, or skin patch 2, on a selected treatment area. In this embodiment, a skin patch 2 is applied over the skin 1, where a specific area has been determined to be a desired treatment area 3. Prior to exposure to laser light 5 emitted from the treatment laser 4, the skin patch 2 is transmissive, or substantially transmissive, to the light 5 emitted by the treatment laser 4.

FIG. 2 illustrates an application of the skin patch 2a where the skin patch 2a has optically changed. The optical change has occurred as a result of the heating of the skin patch 2a, due to exposure to laser light 5 emitted by the laser 4. In this embodiment, the skin patch 2a has changed optically, or activated, as a result of the heating in the treatment of the treatment area 3.

Although referred to herein as laser light 5, it is recognized that photodynamic therapy may involve other energy sources, any one of which may be referred to as “therapeutic light 5.” The skin patch 2 therefore is not functional only for laser light 5, but any wavelength of light or other energy form to which the thermochromic materials within the skin patch 2 may be sensitized. The term “photodynamic therapy” is therefore considered herein to encompass any therapeutic procedure that results in localized heating of tissue through use of an optical source, and provides an opportunity for use of the teachings disclosed herein to limit temperature increases. Therefore, as used herein “photodynamic therapy” is synonymous with “an optically based therapeutic process”, and such a procedure is not limited by other aspects of conventional photodynamic therapy, such as the introduction of photo-sensitizing agents into the tissue of the treatment area.

In preferred embodiments, the skin patch 2a is formed of temperature sensitive materials that change from substantially optically transmissive to substantially optically non-transmissive, once a first predetermined temperature, or a lower critical solution temperature (LCST), has been surpassed. As used herein, “substantially optically transmissive” means that the temperature sensitive material, and any other appropriately designated materials, permit adequate transmission of therapeutic light 5 for the completion or undertaking of effective treatment. In contrast, “substantially optically non-transmissive” means that the temperature sensitive materials, and any other appropriately designated materials, effectively reduce or attenuate the therapeutic light 5 reaching the treatment area 3 in order to provide for reductions in energy deposition in the treatment area 3. Attenuation or reduction in the therapeutic light 5 may result from mechanisms such as, and not limited to, absorption, scattering or reflection of the therapeutic light 5 by the temperature sensitive materials.

Materials known to exhibit optical changes once a LCST has been surpassed include various polymers, and mixtures of polymers. Preferred embodiments of the invention disclosed herein make use of temperature sensitive materials exhibiting reversible behavior. That is, as the thermal energy absorbed by the temperature sensitive materials dissipates and temperature of the materials decreases below a second predetermined temperature, the temperature sensitive materials again become substantially optically transmissive. The first and the second temperatures are typically about the same in value, however, the first and the second temperatures may vary from each other at least slightly.

Specific examples of polymer containing materials that may be employed with this invention are contained in the following table, included herein for purposes of illustration only, and are not intended to be limiting of the invention, or any embodiment thereof, unless specified. The table discloses combinations of polymers that have been observed to exhibit LCST behavior. Specific comments are included regarding various formulations.

| POLYMER I | POLYMER II | Comments |
|---|---|---|
| Acrylonitrile-co-αα-methylstyrene | n butyl methacrylate-co-methyl | I was 30 wt % acrylonitrile, II was 70 wt % |
| | methacrylate | methyl metracrylate |
| | ethyl methacrylate | I was 30 wt% acrylonitrile |
| | ethyl metracrylate-co-methyl metracrylate | I was 30 wt % acrylonitrile; II was 30 or 60 |
| | | wt % methul metacrylate |
| | methyl methacrylate | I was 30 wt % acrylonitrile; II was a atactic |
| | | or isotactic |
| Acrylonitrile-co-styrene | εε-caprolactone | I was 28% acrylonitrile |
| | methyl methacrylate | I was 28% acrylonitrile |
| Bisphenol A carbonate; (oxycarbonyloxy- | εε-caprolactone | — |
| 1,4-phenylene isopropylidene-1,4- | | |
| phenylene) | | |
| Butyl acrylate | Chlorinated ethylene | — |
| | Vinyl chloride | — |
| Butyl methacrylate | 2 (hydroxy hexa- | II was 90.3-90.8 mol % styrene |
| | fluorosoisopropyl)styrene-co-styrene | |
| εε-caprolactone | Chlorinated ethylene | II was 30 wt % Cl |
| Carbon monoxide-co-ethyl | Vinyl chloride | I was 13.8/7.41/78.8//carbon |
| acrylate-co-ethylene | | monoxide/ethyl acrylate/ethylene |
| Cellulose acetate | 4 vinylpyridine | I was 10H/2 glucose |
| Chlorinated ethylene | Ethylene co-vinyl acetate | I was 35.4-52.6 wt % Cl; II was 40-45 wt % |
| | | vinyl acetate |
| | Methyl methacrylate | I was 5052 wt % Cl |
| Chlorinated isoprene | Ethylene-co-vinyl acetate | I and II were commercial samples |
| Chlorinated vinyl chloride | Chlorinated vinyl chloride | I and II differed in composition by 3-4% |
| | | Cl |
| | Vinyl chloride | I was ≦≦61.3% Cl |
| o-chlorostyrene | Styrene | — |
| o-chlorostyrene-co-p-chlorostyrene | 2,6-dimethyl-1,4-phenylene oxide styrene | — |
| | | I was 71-92 mol % ortho isomer |
| o-chlorostyrene-co-o-fluorostyrene | 2,6-dimethyl-1,4-phenylene oxide | I was about 14-40 mol % ortho-chloro |
| | | isomer |
| p-chlorostyrene-co-o-fluorostyrene | 2,6-dimethyl-1,4-phenylene oxide | I was 66-74 mol % para isomer |
| Chlorosulfonated ethylene | Vinyl chloride | I was 1% S as SO₂Cl, 42 wt % Cl |
| 2,6-dimethyl-1,4-phenylene oxide | o-fluostyrene-co-p-fluorostyrene | II was 10-38% para isomer |
| | o fluorostyrene-co-styrene | It was 9-20 mole % styrene |
| | p fluorostyrene-co-styrene | II was about 22-54 mol % styrene |
| Dodecamethylene decamethylene | Vinyl chloride | — |
| dicarboxylate | | |
| Dodecamethylene dodecamethylene | Vinyl chloride | — |
| dicarboxylate | | |
| Ethyl acrylate | Vinylindene fluoride | — |
| Ethyl methacrylate | 2-(hydroxy-hexafluoro-isopropyl) styrene- | II was 90.3-98.9 mole % styrene |
| | co-styrene | |
| | vinyl chloride-co-vinylidene chloride | II was 86.5 wt % vinylidene chloride |
| | vinylidene fluoride | I was syndiotactic or atactic; LCST below |
| | | m.p. of II if I was high mol. Wt. Isotactic |
| Ethylene-co-vinyl acetate | Vinyl chloride | I was 30 or 37 wt % ethylene |
| Ethylene oxide | Oxyphenylene-sulfonyl-phenylene | — |
| Hexadecamethylene dodecamethylene | Vinyl chloride | — |
| dicarboxylate | | |
| 2-(hydorxy-hexafluoroisopropyl) styrene- | Methyl methacrylate | I was 90.3-96.1 mole % styrene |
| co-styrene | | |
| | Vinyl methyl ether | I was 90.3-99.9 mole % styrene |
| Methyl acrylate | Vinylidene fluoride | — |
| Methyl methacrylate | Vinyl chloride | I was atactic |
| | Vinyl chloride-co-vinylidene chloride | I was atactic or sitactic; II was 86.5 wt% |
| | | vinylidene chloride |
| | Vinylidene fluoride | LCST above decomposition T of I |
| Neopentyl adipate | Oxy-2-hydroxytrimethylene-1,4- | — |
| | phenyleneisopropylidene-1,4- | |
| | phenylene; (Phenoxy resin) | |
| | Vinyl chloride-co-vinylide chloride | II was Saran 86.5 wt % vinylidene |
| | | chloride LCST above m.p. when ≦≦50 |
| | | wt % I |
| Oxycarbonyloxy-2,6-dimethyl-1,4- | Styrene | — |
| phenyleneisopropylidene-3,5-dimethyl- | | |
| 1,4-phenylene | | |
| n-propyl methacrylate | Vinyl chloride-co-vinylidene chloride | II was 86.5 wt % vinylidene chloride |
| Styrene | Vinyl methyl ether | I was hydrogenated or deuterated |
| Vinyl methyl ketone | Vinylidene fluoride | — |

In other embodiments, other thermochromic materials are used within the skin patch 2a.

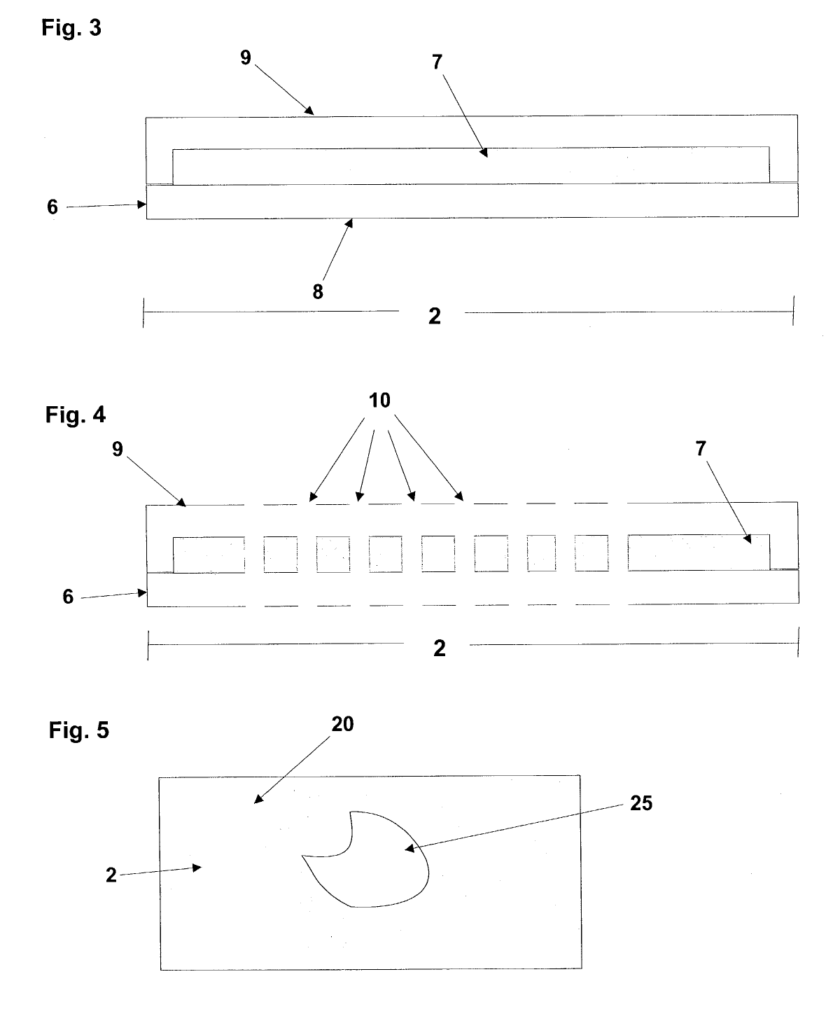

FIG. 3 is an illustration of a preferred embodiment of the skin patch 2. In this embodiment, a temperature sensitive material 7 is layered on top of a backing 6. The underside 8 of the backing 6 may be coated with an adhesive used to affix the skin patch 2 in place over the treatment area 3. The temperature sensitive material 7 that is applied over the backing 6 may be protected by a protective layer 9. This protective layer 9 prevents abrasion or other types of loss of the temperature sensitive material 7, thereby preserving the reliability of the skin patch 2 temperature response.

The skin patch 2 is constructed in a manner that provides for optimal dissipation of heat from the treatment area 3. FIG. 4 shows a preferred embodiment, where in the skin patch 2 is perforated with multiple perforations 10, thereby permitting heat loss through natural perspiration.

In other embodiments, a customized skin patch 2 is provided for treatment. An example is shown in FIG. 5. In FIG. 5, the layer of temperature sensitive material 7 is distributed over an area 20 that correlates to the healthy tissue surrounding a treatment area 25. The treatment area 25 therefore being exposed by a “cut-out” in the layer of temperature sensitive material 7.

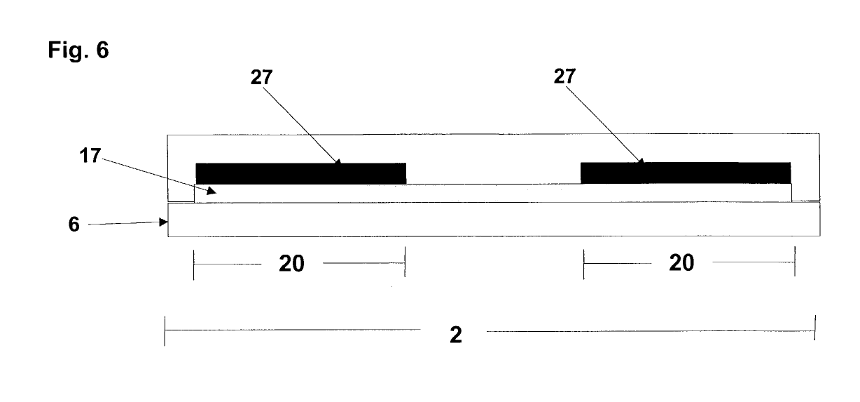

Further, in view of the embodiment shown in FIG. 5, it can be understood that multiple layers of temperature sensitive materials 7 can provide additional benefits. For example, in one embodiment, shown in FIG. 6, a first layer of temperature sensitive material 17 is incorporated into an entire patch 2 and changes optically as described herein at a predetermined temperature. A second layer of temperature sensitive materials 27 is incorporated into the patch 2 over only those areas 20 known to be healthy. The second layer 27 exhibits an optical change as described herein at a predetermined temperature that is lower than that of the first layer 17.

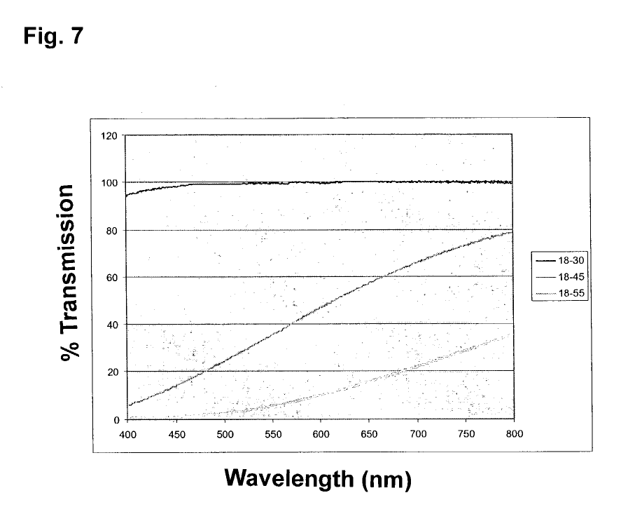

Embodiments, and aspects thereof, of exemplary formulations of LCST materials are now introduced. A first embodiment, referred to as “formulation A” was produced from a mixture of 6.76 grams of 60% aqueous polyvinylmethylether (PVME), 0.56 g Water, 1.46 g 25% aqueous sodium dodecyl sulfate (SDS), and 1.22 g polyethylene glycol diacrylate (having a molecular weight of about 575). A set of typical transmission curves for formulation A is presented in FIG. 7.

In FIG. 7, percent transmission is presented as a function of wavelength. The transmission was assessed at 30° C., 45° C. and 55° C. FIG. 7 shows a progressive decrease of percent transmission with an increase in temperature. The drop in transmissivity with increasing temperature is shown over the entire range of wavelengths evaluated.

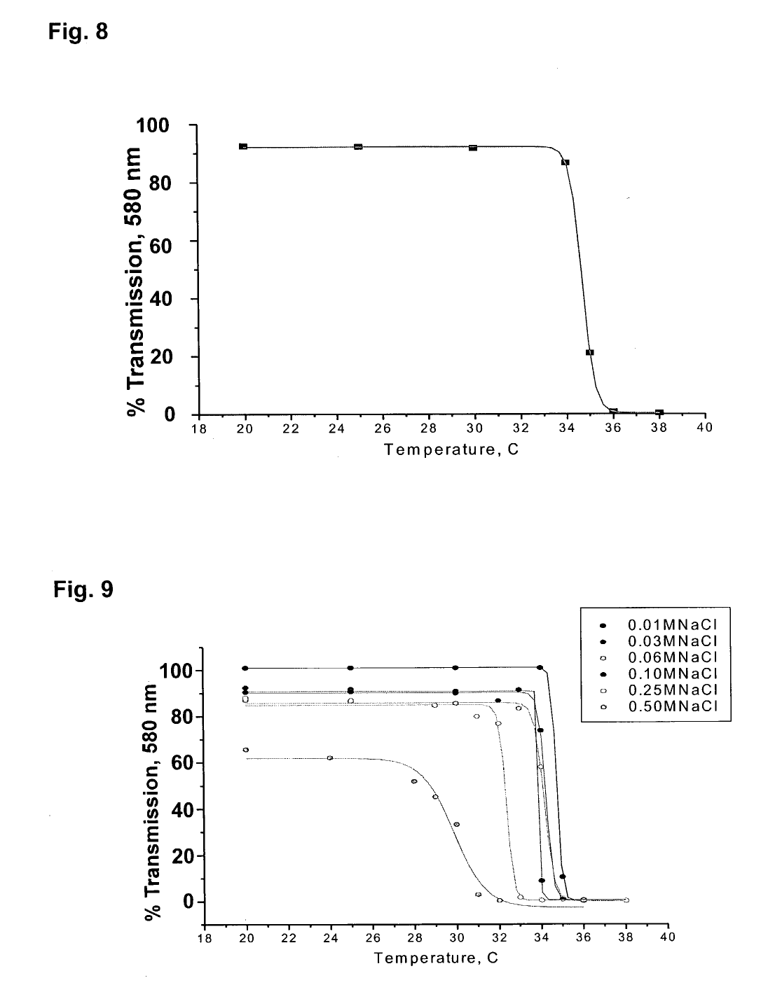

In order to produce a formulation offering improved temperature transition characteristics, a series of samples were produced. The performance of these samples is shown in the graphs of FIGS. 8-11.

FIGS. 8-11 show the behavior of a screening formulation used to exemplify tuning a formulation for temperature transmission. Mixtures of 80% aqueous PVME, 7.5% polyethyleneglycol diacrylate (having a molecular weight of about 200) (SR-259™ of Sartomer Corporation) containing 5% oligo(2-hydroxy-2-methyl-1,4(1-methylvinyl)phenyl)propanone and 2-hydroxy-2-mehtyl-1-phenyl-1-propoanone (KIP100F™ also from Sartomer Corporation), 2.5% SDS (25 wt % aqueous), and 10% of water, solvent, and/or salt were combined to achieve the indicated concentrations.

Samples of the mixtures were sandwiched in a 0.20 mm quartz cuvette and cured by exposure on each side to four seconds of light produced by a Xenon RC-250™ (“c” bulb) lamp (from Xenon Corporation of Woburn Mass.). A custom transmission cell holder with a thermoelectric stage was used to control the temperature during subsequent measurements. The transmission spectra from 400 nm to 800 nm were recorded at 20° C., 25° C., 30° C., and 35° C., and again at 20° C. after cooling to assure the reversibility of the transition. Graphs of transmission at 580 nm for each additive are provided. The control samples showed excellent behavior, with a transition temperature of 35° C., as seen in FIGS. 8-11.

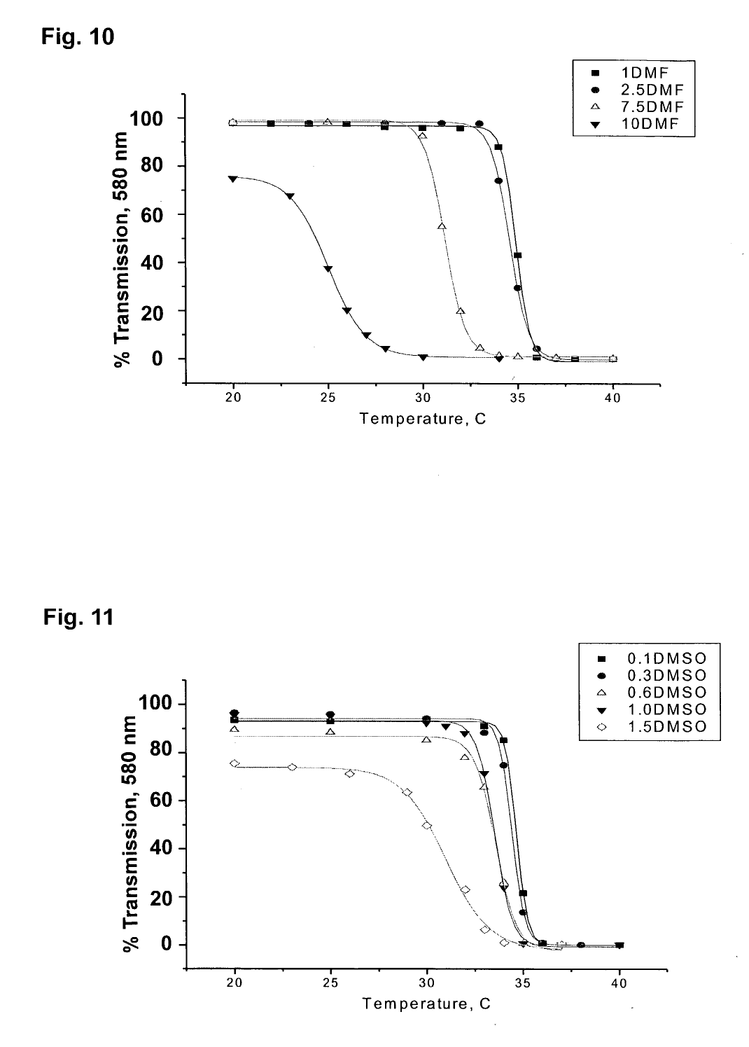

FIG. 8 depicts the optical transmission transition as a function of temperature for the foregoing mixture. FIG. 9 further depicts the optical transmission transition as a function of temperature for the foregoing mixture with the addition of various concentrations of NaCl. FIG. 10 provides similar information wherein concentrations of and additive dimethyl formamide (DMF) are varied. FIG. 11 shows similar data for varied concentrations of dimethylsulfoxide (DMSO).

FIGS. 9-11 show that the additives appear to give excellent results in establishing a process for tuning the temperature response of the specific mixture, without severe broadening of the transition or reduction of low temperature clarity.

An exemplary embodiment of material suited for use in a skin patch 2 was produced using formulation A. Using this mixture, a four inch square was manufactured using masking tape applied onto a substrate of polypropylene film. A 10 mil think layer of formulation A from above was coated onto the film across the mask using an adjustable doctor blade coater. The masking tape was removed leaving a square area of gel on the film. The coating was crosslinked by exposure to an RC747™ pulsed Xenon lamp (“c” bulb) for one second. A second layer of polypropylene film was then laminated onto the top of the gel, and heat sealed around the gel square to form a sealed laminate. In high volume production, squares of formulation A, or similar material disclosed herein, or developed in accord with the teachings herein, may be printed using printing methods such as, and not limited to, screen printing or gravure methods.

Other materials that are suited for use as a substrate include, and are not limited to, BOPP, Daran, PET and other clear plastic films. Preferred characteristics for a substrate include having a high degree of optical transparency at the wavelength used for the photodynamic therapy, providing an effective barrier against migration of water to limit water loss from the temperature sensitive materials, and having a thickness as thin as possible to limit interference with heat transmission from the treatment area 3.

Further examples of methods and apparatus for the production of a layer of temperature sensitive materials 7 suited for practice of this invention are now provided.

Forming a Gel that Exhibits LCST Behavior

Gels are defined herein as semisolid systems in which the movement of the dispersing medium is restricted by an interlacing network of particles or solvated macromolecules of the dispersed phase. The gels formed by crosslinking the formulations disclosed herein are of a type in which the macromolecules are uniformly distributed throughout a liquid with no apparent boundaries between the dispersed macromolecules and the liquid. Upon heating through the LCST transition point of the solution (gel), the gel changes to a two phase system in which the gel mass contains a suspension of small distinct particles capable of effectively scattering light. The result is a mechanically robust film capable of mimicking the LCST behavior of the solution from which it is derived.

The formulation includes an aqueous solution of a polymer exhibiting LCST behavior, such as poly(methylvinyl ether), or PVME. The formulation then also includes a water dispersible crosslinking agent, such as poly(ethylene glycol) diacrylate and a crosslinking initiator such KIP100F™, of Sartomer Corporation. A number of other water dispersible crosslinking agents and initiators are commercially available. A surfactant is preferably then used to modify the LCST transition temperature, as well as the particle size and opacity of the materials after transition. The ratios and composition of the LCST polymer, water, crosslinking agent, initiator, and surfactant can all be adjusted to achieve the desired properties. These solutions and the subsequent gels can be optimized for various desired properties including, but not limited to, viscosity, mechanical strength, adhesion, opacity and the LCST transition point.

The prepared LCST solution may then be coated onto a variety of substrates, and often exhibit significant adhesive properties suitable for bonding two substrates. Therefore, in one embodiment of the formation of a LCST gel, the LCST solution is sandwiched between two substrates and subsequently cured to form a robust laminate structure. The laminate structure then exhibits LCST behavior as the structure is heated through the gel LCST transition point. The substrates can be chosen from a variety of transparent or opaque materials including glasses, thermoplastics, metals, and ceramics. A presently preferred, but not limiting, embodiment includes thin, transparent labels having a conventional clear, adhesive-backed film on one side, an application of LCST solution, and a clear film on the opposite side.

In a preferred and non-limiting embodiment, formulation of a gel exhibiting LCST behavior (herein LCST gel) comprises an aqueous solution of a material exhibiting LCST behavior, a water dispersible crosslinking agent, a crosslinking initiator and may include a surfactant. The LCST material may be comprised of poly(methylvinyl ether) or hydroxypropylcellulose. The LCST material may be selected from the family of N-alkyl acrylamides. The water dispersible crosslinking agent may be comprised of poly(ethylene glycol) diacrylate, or ethoxylated trimethylolpropane triacrylate, or ethoxylated bisphenol A diacrylate.

The crosslinking initiator may be comprised of a eutectic mixture of 2,4,6-trimethylbenzophenone and 4-methylbenzophenone (TZ™); a blend of phosphine oxide, alpha-hydroxy ketone and a benzophenone derivative (KTO46™); and a blend of phosphine oxide, alpha-hydroxy ketone and a benzophenone derivative (KIP100F™). The materials known as TZT™, KTO46™, or KIP100F™ are commercially available from Sartomer Corporation.

The surfactant may be comprised of sodium dodecyl sulfate, sodium dioctyl sulfosuccinate, or an amine salt of a branched or linear alkylbenzenesulfonic acid.

The aqueous solution of a polymer exhibiting LCST behavior exhibits a change in optical transmission of visible light of at least 20% upon transition, preferably 50%, and most preferably 90% or greater. The viscosity of the aqueous solution of the polymer exhibiting LCST behavior before crosslinking is below 100,000 cP, preferably below 50,000 cP, and most preferably below 10,000 cP.

In another embodiment, a film formed from the LCST gel may exhibit adhesive properties making it suitable for application as a laminating adhesive. A laminated article can be comprised of the formulation given above and at least two supporting sheets, where the supporting sheets can be comprised of plastic, glass, metal or ceramic.

What follows are presently preferred, but not limiting, examples of formulations for a LCST gel. Included is an example of a process for fabrication of a laminated layer of temperature sensitive materials 7. In various embodiments, the LCST may display other desirable properties in addition to reversible opacity, including but not limited to, adhesive properties.

A plurality of preferred embodiments of aqueous solutions of polymers that can be crosslinked into mechanically robust gels that exhibit reversible LCST behavior is disclosed in Table 1, however, this list is not limiting. Table 1 provides a series of mixtures of poly(methylvinyl ether) ((60 wt % in water, PVME), water, sodium dodecyl sulfate 25 wt % in water, SDS), and a crosslinking mixture (PEG-575) of poly(ethylene glycol) diacrylate (molecular weight of approximately 575) and 5% KIP100F™ were prepared by manual stirring. The resulting solutions were all transparent in the visible range of 400-800 nm (>95% transmission versus water) at 30° C. or below. Transmission measurements in this range were recorded at 45° C. and 55° C. versus a water reference. Table 1 summarizes the composition and transmission data at 500 nm for each example. The transition temperature was recorded as the temperature at which the film became visibly white.

TABLE 1

| | Components, grams | | | | Gel Properties | | |
|---|---|---|---|---|---|---|---|
| Example # | 60% PVME | Water | 25% SDS | PEG-575 | Transition T (° C.) | % T, 45° C. | % T, 55° C. |
| 1 | 6.5 | 1.167 | 0.833 | 1.5 | 40 | 47.163 | 18.418 |
| 2 | 7.5 | 0.667 | 1 | 0.833 | 41 | 25.166 | 0.63148 |
| 3 | 6 | 1 | 2.5 | 0.5 | 46 | 57.3 | 29.048 |
| 4 | 6 | 3 | 0.5 | 0.5 | 39 | 5.1657 | 3.0294 |
| 5 | 6 | 0 | 2.5 | 1.5 | 46 | 80.211 | 37.285 |
| 6 | 7.5 | 1.5 | 0.5 | 0.5 | 38 | 1.1463 | 0.52417 |
| 7 | 6 | 2.25 | 1.25 | 0.5 | 41 | 15.631 | 1.3861 |
| 8 | 7.5 | 0 | 1 | 1.5 | 44 | 81.528 | 54.809 |
| 9 | 6 | 2 | 0.5 | 1.5 | 39 | 40.407 | 5.4845 |
| 10 | 7.5 | 0.5 | 0.5 | 1.5 | 44 | 53.926 | 1.6308 |
| 11 | 6 | 1.167 | 1.667 | 1.167 | 42 | 54.391 | 15.957 |
| 12 | 7.5 | 0 | 2 | 0.5 | 44 | 6.3153 | 0.85948 |
| 13 | 6.8 | 1.3 | 1.4 | 0.5 | 43 | 4.7153 | 0.55757 |
| 14 | 6.5 | 0 | 2.5 | 1 | 46 | 54.945 | 15.918 |
| 15 | 6.75 | 2.25 | 0.5 | 0.5 | 39 | 1.2005 | 1.5455 |
| 16 | 6.75 | 1.25 | 0.5 | 1.5 | 40 | 29.175 | 1.2005 |
| 17 | 6.764 | 1.931 | 0.583 | 0.722 | 39 | 2.6042 | 3.475 |
| 18 | 6.764 | 0.556 | 1.458 | 1.222 | 43 | 24.486 | 2.6029 |
| 19 | 6 | 2 | 0.833 | 1.167 | 41 | 14.057 | 1.0338 |
| 20 | 7.5 | 0 | 2 | 0.5 | 41 | 1.6818 | 0.44653 |

The LCST transition preferably occurs slightly above body temperature (37° C.), or the surface temperature of the treatment area 3. Therefore, Example 6 having a transition temperature of 38° C., Examples 4, 9 15, and 17 having transition temperatures of 39° C., and Example 16 having a transition temperature of 40° C. are suited for certain embodiments of the layer of temperature sensitive materials 7. That is not to say that the other Examples, or other materials disclosed herein, are not suited for practice of this invention. In fact, the methods disclosed herein for tuning the transition temperature, can be used to improve characteristics of these or other embodiments of temperature sensitive materials.

Other preferred embodiments of aqueous solutions of polymers that can be crosslinked into mechanically robust gels that exhibit reversible LCST behavior are disclosed in Table 2. In Table 2, the use of different crosslinking materials is illustrated.

The crosslinking materials disclosed in Table 2 include triacrylates (such as SR9035™ SR259™ and SR415™, available from Sartomer Corporation), poly(ethyleneglycol) diacylates (with molecular weights of 200 or 575 (referred to herein as PEG200 and PEG575, respectively)) and three crosslinking initiators (TZT™, KTO46™, and KIP100F™). These examples were prepared by mixing 8.0 grams of 50 wt % PVME in water, 1.0 g of 25 wt % sodium dodecyl sulfate in water, and 1.0 g of the crosslinking materials identified in Table 2. The results are summarized in Table 2.

TABLE 2

| | | Transition | % T, 500 nm | |
|---|---|---|---|---|
| Example # | Crosslinker | Temperature, C. | 45 C. | 55 C. |
| 21 | 5% TZT in SR9035 | 41 | 1.11 | 0.85 |
| 22 | 5% TZT in PEG575 | 41 | 0.76 | 0.63 |
| 23 | 5% TZT in PEG200 | 42 | 0.61 | 0.68 |
| 24 | 5% KTO46 in SR9035 | 40 | 0.49 | 0.46 |
| 25 | 5% KTO46 in PEG575 | 41 | 1.47 | 0.55 |
| 26 | 5% KTO/46 in PEG200 | 40 | 0.71 | 0.56 |
| 27 | 5% KIP100F in SR9035 | 41 | 0.76 | 0.54 |
| 28 | 5% KIP100F in SR415 | 42 | 7.08 | 0.74 |
| 29 | 5% KIP100F in SR259 | 40 | 0.46 | 0.47 |
| 30 | 5% KIP100F in PEG575 | 42 | 1.95 | 0.47 |
| 31 | 10% KIP100F in SR9035 | 42 | 0.57 | 0.49 |
| 32 | 10% KIP100F in PEG575 | 41 | 0.84 | 0.42 |
| 33 | 10% KIP100F in PEG200 | 40 | 0.52 | 0.58 |

In a further embodiment, an 80 g of a solution of PVME (50 wt % in water) is combined with 10 grams of an amine alkylbenzene sulfonate (distributed as NINATE™ 411 by Stepan Corporation), 9.5 g of poly(ethylene glycol) diacrylate (with a molecular weight of approximately 575), and 0.50 grams of KIP100F™ to yield a viscous clear solution that exhibits a LCST transition at approximately 38° C. For the sake of further reference, this material is referred to herein as “material 34.”

In one embodiment, material 34 is sandwiched between plastic films and crosslinked by exposure to UV irradiation from a UV source (250-370 nm) to yield a gel that exhibits a LCST transition at approximately 38° C. In another embodiment, material 34 is deposited upon a single plastic film, and crosslinked by exposure to UV irradiation from a UV source (250-370 nm) to yield a gel that exhibits a LCST transition at approximately 38° C.

If desired, the crosslinked gel can be removed from the plastic film(s) for subsequent use as a film. In this embodiment, the film formed from material 34, once removed from the sandwich, is rubbery in appearance or feel.

The use of the technique described for manufacture of a film from material 34 is not limiting in that variations of LCST materials, curing stimulus, and sandwich support materials as disclosed herein are not intended to be limiting. For example, a further variation is now provided.

In a further embodiment of formation of a film of LCST material, a sample of material 34 was used to adhere two sheets of about two mil (0.05 mm) polyester together by roll lamination using an approximately 0.2 mm spacer between the polyester sheets. The resulting article was approximately 0.3 mm in thickness. This laminate was then exposed to UV irradiation to effect crosslinking. The sheets were well adhered and the LCST layer no longer flowed upon application of pressure to the laminate. The laminate structure exhibited reversible opacity change at the LCST transition of the gel (approximately 38° C. in this case). The laminate structure exhibited a change in transmission of visible light from 90% transmissive to <5% transmissive upon LCST induced opacity change.

Color Forming Materials

In other embodiments, the layer of temperature sensitive materials 7 may develop highly visible color or contrast at the temperature transition, thereby becoming readily visible to the person applying the photodynamic therapy. This feature provides for improved manual control over the therapy source.

In order to evaluate methods and apparatus for the development of color in a temperature sensitive material, experiments were conducted.

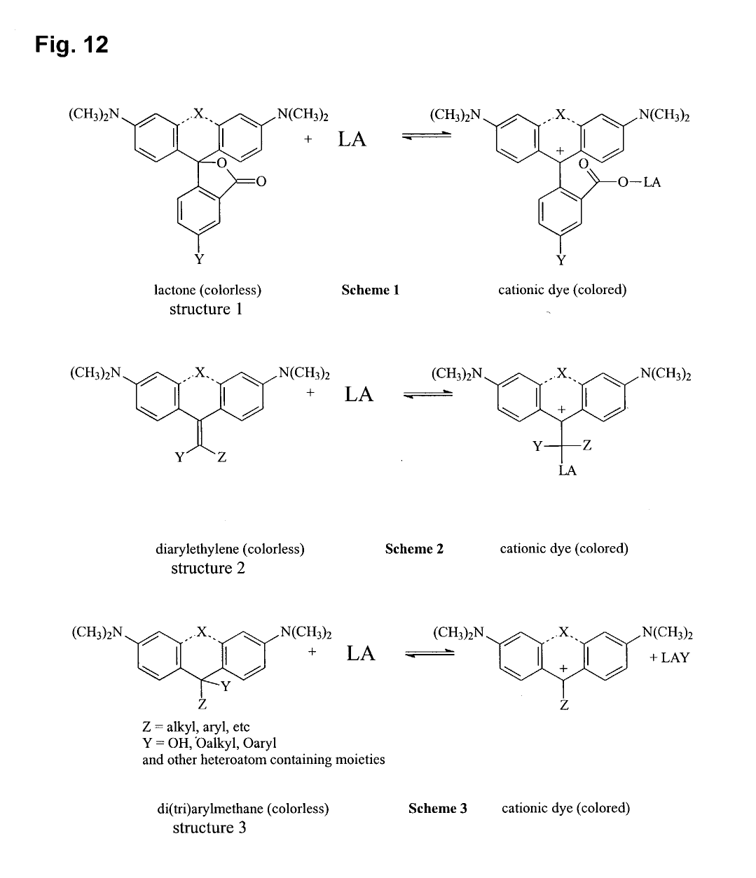

In a first set of experiments, it was reasoned that since the opacity formed upon heating a polymer mixture or solution above the LCST could be caused by phase separation of two or more polymers with differing chemical properties, that the formation of two very different environments could provide a basis to “turn on” a dye. It was reasoned that a color former when combined with a Lewis acid in the presence of an LCST mixture, the Lewis acid would be complexed to the more Lewis basic polymer and would be unavailable to cause formation of the colored form of a color former. It was further reasoned that once heated to the point of phase separation, enough Lewis acid and color former would be left in the less Lewis basic component to cause the formation of the colored form of the color former. In concept, any color former and Lewis acid pair could be used. FIG. 12 shows the chemical structures of three different schemes that were found suited to this invention. A series of combinations were tested through the process described herein.

In order to test color development in a temperature sensitive material as disclosed herein, a LCST polymer solution was made by taking 12 grams of a 50% aqueous solution of poly(methyl vinyl) ether (Aldrich #18.272-9), which was placed in a 200 ml round bottom flask along with 100 ml of benzene. A stir bar was placed in the flask which was then heated in an oil bath. Once reflux was reached, the water was azeotropically removed through use of a water separator equipped with a condenser. Once the water was removed to give a clear benzene solution, toluene was added in portions as the benzene was removed by distillation. In the end, a clear toluene solution containing 6 grams of poly (methyl vinyl) ether dissolved in 50 ml of toluene was obtained. To this solution was added a solution of 4 grams of polystyrene (Aldrich #33,165-1) dissolved in 50 ml of toluene. Aliquots of the binary polymer solution were coated on glass slides and air dried to give clear films with a rubbery texture. Heating the slides over a heat gun (with a temperature ranging from about 100° C. to about 150° C.) caused the clear film to turn opaque white. Upon cooling, the films returned to their clear form.

The color formers were made up in tetrahydrofuran (THF) at a concentration of 50 mg/ml, and the Lewis acids were made up in methanol at 250 mg/ml. To prepare the thermochromic mixture, various amounts of the color former solutions were mixed with varying amounts of the Lewis acid solutions and this was added to 1.0 ml of the polymer solution. The amount of color former used was 20, 40, 60, 80, and 100 μl of the THF solution, (1, 2, 3, 4 and 5 mg of color former) per 1.0 ml of polymer solution and the amount of Lewis acid used was 4, 8, 12, 16 and 20 μl of the methanol solution (1, 2, 3, 4 and 5 mg of Lewis acid) per 1.0 ml of the polymer solution. The Lewis Acid and color former solutions were first mixed together before they were added to the polymer solution. It was found that the best color was formed when the amount of color former was 5 mg and the Lewis Acid was either 3-nitrophenyl boronic acid, 3,4-dichlorophenylboronic acid, 3,5-dichlorophenylboronic acid, 3,5-difluorophenylboronic acid or pentafluorophenylboronic acid in the amount of 5 mg per each 1.0 mL of polymer solution. When these mixtures were spotted on a glass plate and air dried, a colorless clear polymer film formed which was had a rubbery texture. These films, when heated became intensely colored and faded quickly over a few minutes back to the original colorless form upon cooling. The exceptions were the films made with rhodamine B base, 2,2-bis-(4-dimethylaminophenyl)-1,3-dithiolane and leucocrystal violet cyanide. The room temperature film with rhodamine B base was light pink in color which became intensely red upon heating. Upon cooling the film slowly returned to the pink coloration over about 30 minutes. Changing the Lewis acid would probably have an effect on the color of the room temperature film as well as the time needed to return to the original color. The films made with 2,2-bis-(4-dimethylaminophenyl)-1,3-dithiolane and leucocrystal violet cyanide irreversibly turned colored with heating. The cycle of heating to produce color and cooling to return to the colorless form could be repeated numerous times with no apparent loss of efficiency in the case of the reversible films.

Experimentation with protic acids such as benzoic acid and pyridinium p-toluenesulfonate was inconclusive, however, promising. It is considered that appropriate choices of protic acid, color former, and LCST polymer mixture could possibly make this system work with improved efficiency.

Color formers found to be operable in this system include lactone color formers, di(tri)aryl methane carbinol and ether color formers and the diarylethylene color formers. Lewis acids that are operable in this system include any of those found in carbonless copy papers such as phenols, metal ions and boronic acids.

Specific examples of color formers and Lewis acids that fulfill the requirements of this invention are contained in the following table, included herein for purposes of illustration only, and are not intended to be limiting of the invention, or any embodiment thereof, unless specified.

| COLOR FORMER | LEWIS ACID |
|---|---|
| Scheme I: | 3-nitrophenylboronic acid |
| Crystal violet lactone | 3,4-dichlorophenylboronic acid |
| [structure 1, X = H, H and Y = | |
| N(CH3)2] | |
| Rhodamine B base (RBB) | 3,5-dichlorophenylboronic acid |
| [structure 1, X = H, H and Y = H] | |
| Malachite green lactone | 3,5-difluorophenylboronic acid |
| [structure 1, X = O and Y = H] | |
| | pentafluorophenylboronic acid |
| Scheme II: | 4-fluorophenol |
| 1,1-(4-dimethylaminophenyl)ethylene | 2,4-di-t-butylsalicylaldehyde |
| [structure 2, X = H, H and Y = Z = H] | |
| | 3-methoxyphenylboronic acid |
| Scheme III: | 4-fluorophenylboronic acid |
| 2,2-bis-(4-dimethylaminophenyl)-1,3- | 4-chlororphenylboronic acid |
| dithiolane [structure 3, X and Y | |
| together = —SCH2CH2S—] | |
| Leucocrystal violet cyanide (LVC) | 2,4-difluorophenylboronic acid |
| [structure 3, X = | |
| H, H, Y = CN, and Z = 4 | |
| dimethylaminophenyl] | |
| | 9-hydroxyboroxarophenanthrene |

Of the Lewis Acids tested, it was found that 3-nitrophenylboronic acid, 3,4-dichlorophenylboronic acid 3,5-dichlorophenylboronic acid, 3,5-difluorophenylboronic acid and pentafluorophenylboronic acid worked the best. It was noted that the pentafluorophenyl boronic acid worked well with crystal violet lactone, but was relatively ineffective with malachite green lactone. The 3-nitrophenylboronic acid worked with both crystal violet lactone and malachite green lactone.

A second series of LCST polymer mixtures were synthesized in a manner similar to the process as described in Example #2 of U.S. Pat. No. 4,722,595, issued Feb. 2, 1988 to Siol, and entitled “Process for Displaying Optically Readable Information. Therefore, Example 2 of U.S. Pat. No. 4,722,595, and the related teachings, are incorporated by reference herein in their entirety.

Four separate trails were completed in the second set of experiments. These trials are referred to as Run #1, Run #2, Run #3 and Run #4. Run #1 was performed under argon and combined 50 grams of chlorinated polyisoprene (Aldrich #46,252-7), 100 grams of 2-ethylhexyl methacrylate, 100 grams of isobutyl methacrylate and 3 mL of a 75% solution in mineral spirits of t-amyl peroxy-2-ethylhexanoate (Lupersol 575-M75 from Atofina Chemicals of Philadelphia, Pa.). This mixture was heated at bout 70° C. to about 80° C. for approximately six hours. The resulting polymer solution was viscous with a 64.8% solids content. Run #2 followed the steps taken in Run #1, with the addition of 0.5 mL of n-dodecylmercaptan as a chain transfer agent. The resulting polymer solution in Run #2 was appreciably less viscous than Run #1 and contained 57.8% solids. Run #3 used the same amounts of reactants as Run #1, but the reaction was completed without argon protection and only 1.5 mL of Lupersol 575-M75 as the initiator. This polymer solution was the least viscous of the three runs and contained 47.9% solids. Run #4 used the same amounts of reactants as #1, with the exception that the amount of isobutylmethacrylate was reduced to 50 grams. In addition, the reaction in Run #4 was completed without argon protection, and having 1.5 mL of Lupersol 575-M75 as the initiator. All four solutions made clear rubbery films that phase separated upon heating. The polymer from Run #4 had the lowest LCST.

It was found that the addition of crystal violet lactone and 3-nitrophenylboronic acid, both at a concentration of 5 mg/mL of polymer solution, produced a colorless polymer solution. When spread on a glass plate and dried, a clear, colorless film resulted. Upon warming to the point of phase separation, a dark blue color formed. Cooling back to room temperature caused the film to become colorless again. In further experimentation, sucrose diacetate hexaisobutyrate (SAIB, Acros #41978-0010) was tried as a plasticizer since it is a glass at room temperature and becomes free flowing at about 60° C. The SAIB could be dissolved in toluene and than added to the polymer solutions. When these mixtures were dried, a rubbery, clear film was formed even when the SAIB content was 50% or greater. Heating the films caused them to become viscous and phase separate.

The experimentation confirmed that color formers in combination with Lewis acids and properly introduced into a LCST material may be selected for use in the layer of temperature sensitive materials 7. These combinations provide for a layer of temperature sensitive material 7 that is substantially optically transmissive, or transparent, below a threshold temperature (LCST), while becoming colored and absorbing above the threshold temperature.

As another example, it is considered that the layer of temperature sensitive materials 7 may be applied in a form other than a patch 2, such as by application of the temperature sensitive materials in an appropriately formulated composition of matter. The composition of matter may be in the non-limiting forms of a paint or a cream for applying directly to the skin, or applied over some suitable intermediary substance, such as a transparent substrate, or another possibly non-optically active transparent cream or paint. The paint or cream may be based in a carrier suited for treatment of the target tissue. For example, a cream could be based on certain moisturizers, emollients or other ingredients known for dermatologic treatment or care. In other embodiments a medication, such as a topical anesthetic, may be included to enhance patient comfort, or otherwise enhance treatment.

In embodiments where the layer of temperature sensitive materials 7 are applied as a paint, the paint could be applied over a treatment area 3 by conventional techniques, such as brushing. The paint could be air dried, cured by exposure to a light, or otherwise cured. The paint may include further materials as necessary to provide for curing. In further embodiments, the paint is applied over an intermediary device, such as a balloon, or other suitable jig. Once cured, the paint is removed from the intermediary device, and applied to the treatment area as the patch.

Where the temperature sensitive materials are applied as a cream, paint, or other form, the form applied exhibits the desired properties of providing a form that is generally substantially optically transmissive and further becomes substantially optically non-transmissive above a predetermined threshold temperature.

Certain LCST materials such as aqueous solutions of hydroxypropylcellulose (HPC), offer the opportunity to formulate gels that may be applied directly to the skin. For examples, solutions containing approximately 10% to 30% HPC by weight in water, may be formulated with other materials such as aloe vera, safflower oil, glycerin, jojoba oil, borage oil, and fatty acids, esters, and alcohols commonly found in skin care products to form a transparent gel which can be directly applied to the skin. Improved temperature sensitivity and control may be realized in certain embodiments where a cream or paint is applied directly onto the skin.

One skilled in the art will recognize that the invention disclosed herein is not limited to the foregoing embodiments. For example, it is considered that other formulations of temperature sensitive materials may be used to practice the teachings disclosed herein. Furthermore, it is considered that temperature sensitive materials could be produced on a separate film, as previously described, and subsequently applied directly onto the skin. Therefore, it is considered that the embodiments described herein, and others are within the teachings of this invention.

What is claimed is:

1. A method to limit temperature increases in a treatment area during an optically based therapeutic process that results in the heating of the treatment area, the method comprising:
applying a thermally sensitive material to the treatment area, the thermally sensitive material changing from being substantially optically transmissive to being substantially optically non-transmissive at a predetermined temperature;
exposing the treatment area to therapeutic light; wherein the thermally sensitive material applied to the area changes optically upon surpassing the predetermined temperature due to heating caused by the therapeutic light, thus limiting a further temperature increase in the treatment area.

2. A method as in claim 1, wherein the temperature sensitive material reversibly becomes substantially optically transmissive upon cooling below a second predetermined temperature.

3. A method as in claim 1, wherein the thermally sensitive material is contained in a patch.

4. A method as in claim 1, wherein the thermally sensitive material is contained in at least one of a paint or a cream.

5. A method as in claim 1, wherein the thermally sensitive material comprises at least one polymer.

6. A method as in claim 1, wherein the thermally sensitive material comprises a mixture of water, polymethylvinylether, sodium dodecyl sulfate, and polyethylene glycol diacrylate, the diacrylate having a molecular weight of about 575.

7. A method as in claim 1, wherein the thermally sensitive material comprises a mixture of water, polymethylvinylether, polyethylene glycol diacrylate, the diacrylate having a molecular weight of about 200, oligo(2-hydroxy-2-methyl-1,4(1-methylvinyl)phenyl)propanone and 2-hydroxy-2-mehtyl-1-phenyl-1-propoanone, and sodium dodecyl sulfate.

8. A method as in claim 1, wherein the thermally sensitive material comprises an additive comprising at least one of sodium chloride, dimethyl formamide and dimethylsulfoxide.

9. A method as in claim 5, wherein the temperature sensitive material further comprises at least one colorformer, and at least one Lewis Acid.

10. A method as in claim 9, wherein the colorformer comprises at least one of Crystal violet lactone, Rhodamine B base (RBB), Malachite green lactone, 1,1-(4-dimethylaminophenyl)ethylene, 2,2-bis-(4-dimethylaminophenyl)-1,3-dithiolane, and Leucocrystal violet cyanide (LVC).

11. A method as in claim 9, wherein the Lewis Acid comprises at least one of 3-nitrophenylboronic acid, 3,4-dichlorophenylboronic acid, 3,5-dichlorophenylboronic acid, 3,5-difluorophenylboronic acid, pentafluorophenylboronic acid, 4-fluorophenol, 2,4-di-t-butylsalicylaldehyde, 3-methoxyphenylboronic acid, 4-fluorophenylboronic acid, 4-chlororphenylboronic acid, 2,4-difluorophenylboronic acid, and 9-hydroxyboroxarophenanthrene.

12. A method as in claim 1, wherein the treatment area comprises a portion of epidermis.

13. A patch for use during an optically based therapeutic procedure, the patch comprising:
at least one layer comprising temperature sensitive material, the temperature sensitive material changing being optically substantially transmissive to being substantially optically non-transmissive above a predetermined temperature.

14. The patch of claim 13, wherein the temperature sensitive material reversibly becomes substantially optically transmissive upon cooling below a second predetermined temperature.

15. The patch of claim 13, further comprising an adhesive backing.

16. The patch of claim 13, comprising a protective layer adapted for protecting the temperature sensitive material.

17. The patch of claim 13, comprising at least one perforation for enabling dissipation of heat.

18. The patch of claim 13, wherein the temperature sensitive material comprises at least one polymer.

19. The patch of claim 13, wherein the at least one layer comprises at least one cut-out area.

20. The patch of claim 13, wherein the thermally sensitive material comprises a mixture of water, polymethylvinylether, sodium dodecyl sulfate, and polyethylene glycol diacrylate, the diacrylate having a molecular weight of about 575.

21. The patch of claim 13, wherein the thermally sensitive material comprises a mixture of water, polymethylvinylether, polyethylene glycol diacrylate, the diacrylate having a molecular weight of about 200, oligo(2-hydroxy-2-methyl-1,4(1-methylvinyl)phenyl)propanone and 2-hydroxy-2-mehtyl-1-phenyl-1-propoanone, and sodium dodecyl sulfate.

22. The patch of claim 13, wherein the thermally sensitive material comprises an additive comprising at least one of sodium chloride, dimethyl formamide and dimethylsulfoxide.

23. The patch of claim 18, wherein the temperature sensitive material further comprises at least one colorformer and at least one Lewis Acid.

24. The patch of claim 23, wherein the colorformer comprises at least one of Crystal violet lactone, Rhodamine B base (RIBB), Malachite green lactone, 1,1-(4-dimethylaminophenyl)ethylene, 2,2-bis-(4-dimethylaminophenyl)-1,3-dithiolane, and Leucocrystal violet cyanide (LVC).

25. The patch of claim 23, wherein the Lewis Acid comprises at least one of 3-nitrophenylboronic acid, 3,4-dichlorophenylboronic acid, 3,5-dichlorophenylboronic acid, 3,5-difluorophenylboronic acid, pentafluorophenylboronic acid, 4-fluorophenol, 2,4-di-t-butylsalicylaldehyde, 3-methoxyphenylboronic acid, 4-fluorophenylboronic acid, 4-chlororphenylboronic acid, 2,4-difluorophenylboronic acid, and 9-hydroxyboroxarophenanthrene.

26. A composition of matter for use during photodynamic therapy, the composition of matter comprising:
a carrier adapted for a dermatologic application, the carrier comprising an effective amount of at least one temperature sensitive material, the temperature sensitive material changing the composition of matter from being substantially optically transmissive to being substantially optically non-transmissive above a predetermined temperature.

27. A composition of matter as in claim 26, wherein the temperature sensitive material reversibly becomes substantially optically transmissive upon cooling below a second predetermined temperature.

28. A composition of matter as in claim 26, wherein the composition of matter comprises a cream.

29. A composition of matter as in claim 26, wherein the composition of matter comprises a paint.

30. A composition of matter as in claim 26, wherein the thermally sensitive material comprises a mixture of water, polymethylvinylether, sodium dodecyl sulfate, and polyethylene glycol diacrylate, the diacrylate having a molecular weight of about 575.

31. A composition of matter as in claim 26, wherein the thermally sensitive material comprises a mixture of water, polymethylvinylether, polyethylene glycol diacrylate, the diacrylate having a molecular weight of about 200, oligo(2-hydroxy-2-methyl-1,4(1-methylvinyl)phenyl)propanone and 2-hydroxy-2-mehtyl-1-phenyl-1-propoanone, and sodium dodecyl sulfate.

32. A composition of matter as in claim 26, wherein the thermally sensitive material comprises an additive comprising at least one of sodium chloride, dimethyl formamide and dimethylsulfoxide.

33. A composition of matter as in claim 26, wherein the temperature sensitive material comprises at least one polymer.

34. A composition of matter as in claim 33, wherein the temperature sensitive material further comprises at least one colorformer and at least one Lewis Acid.

35. A composition of matter as in claim 34, wherein the colorformer comprises at least one of Crystal violet lactone, Rhodamine B base (RBB), Malachite green lactone, 1,1-(4-dimethylaminophenyl)ethylene, 2,2-bis-(4-dimethylaminophenyl)-1,3-dithiolane, and Leucocrystal violet cyanide (LVC).

36. A composition of matter as in claim 34, wherein the Lewis Acid comprises at least one of 3-nitrophenylboronic acid, 3,4-dichlorophenylboronic acid, 3,5-dichlorophenylboronic acid, 3,5-difluorophenylboronic acid, pentafluorophenylboronic acid, 4-fluorophenol, 2,4-di-t-butylsalicylaldehyde, 3-methoxyphenylboronic acid, 4-fluorophenylboronic acid, 4-chlororphenylboronic acid, 2,4-difluorophenylboronic acid, and 9-hydroxyboroxarophenanthrene.

37. A composition of matter as in claim 26, further comprising medication for dermatologic application.

38. A patch for application to a tissue during an optically based therapeutic process that results in the heating of the tissue, the patch comprising:
a first area for application over a target tissue, the first area being substantially transparent to a therapeutic light, the patch comprising a second area at least partially surrounding the first area that becomes at least partially non-transmissive to the therapeutic light above a predetermined temperature.
